(19) United States
(12) Patent Application Publication
(10) Pub. No.: US 2014/0163403 A1
(43) Pub. Date: Jun. 12, 2014

(54) AUTOMATED DETERMINATION OF ARTERIAL INPUT FUNCTION AREAS IN PERFUSION ANALYSIS

(71) Applicant: THE TEXAS A&M UNIVERSITY SYSTEM, College Station, TX (US)

(72) Inventors: MARK W. LENOX, College Station, TX (US); QUN LIU, College Station, TX (US)

(73) Assignee: THE TEXAS A&M UNIVERSITY SYSTEM, College Station, TX (US)

(21) Appl. No.: 14/090,352

(22) Filed: Nov. 26, 2013

Related U.S. Application Data
(60) Provisional application No. 61/736,242, filed on Dec. 12, 2012.

(51) Int. Cl. A61B 5/026; A61B 5/00

(52) U.S. Cl. CPC A61B 5/026; A61B 5/7235
USPC 600/504

(57) ABSTRACT

Automatic arterial input function (AIF) area determination is provided that can be used to facilitate the generation of parametric maps for perfusion studies based on various imaging modalities and covering a variety of tissues. Automatic AIF determination can be accomplished by extracting characteristic parameters such as maximum slope, maximum enhancement, time to peak, time to wash-out, and wash-out slope. Characteristic parameter maps are generated to show relationships among the extracted characteristic parameters, and the characteristic parameter maps are converted to a plurality of two-dimensional plots. Automated segmentation of non-AIF tissues and determination of AIF areas can be accomplished by automatically finding peaks and valleys of each phase of AIF areas on the plurality of two-dimensional plots.

### CROSS-REFERENCE TO RELATED APPLICATION

The present application claims the benefit of U.S. Provisional Application Ser. No. 61/736,242, filed Dec. 12, 2012, which is hereby incorporated by reference herein in its entirety, including any figures, tables, or drawings.

### BACKGROUND

Perfusion refers to capillary-level blood flow in tissues and describes the process of blood delivery through capillary beds of a volume of tissue over time. To non-invasively measure tissue perfusion, a tracer is typically injected and an imaging modality such as positron emission tomography (PET), magnetic resonance imaging (MRI), or computed tomography (CT), is used to detect the tracer. Perfusion parametric maps (the correlation of the imaging data to the biological feature or function) are generated using dynamic evaluation curves. A dynamic evaluation curve represents the tracking of the tracer in a certain region along a dynamic imaging sequence as a function of time.

For PET imaging, the dynamic evaluation curve is the time-activity curve (PET-TAC); for MRI imaging, it is the time-intensity curve (TIC); and for CT imaging, it is the time-attenuation curve (CT-TAC). In the various imaging modalities, the dynamic evaluation curves generally involve the tracer kinetics of baseline, wash-in, wash-out and steady state (the “tracer kinetic model”), which are presented according to the imaging modalities, imaging protocols, and tracer properties. A tracer kinetic model can be used to estimate biological parameters through fitting a mathematical model to the dynamic evaluation curve of a pixel or a region of interest (ROI), for example, based on the change of pixel intensities over the dynamic imaging sequence.

The perfusion parametric maps generated by the dynamic evaluation curves of an imaging modality demonstrate blood distribution and tracer clearance rate with parameters such as tissue blood flow (TBF), blood volume (TBV) and mean transit time (MTT). TBF is defined as volume of blood moving through a given vascular network in a tissue per unit time, with a unit of milliliters of blood per 100 g of tissue per minute (ml/min/100 g). TBV is defined as total volume of flowing blood within vascular network, with a unit of milliliters of blood per 100 g of tissue (ml/100 g). MTT is defined as average transit time of all blood elements entering arterial input and leaving at venous output of vascular network, with a unit of second (s).

The quantitative analysis of parametric perfusion maps relies on accurate determination of the Arterial Input Function (AIF), which indicates the concentration of a tracer in a blood pool within blood feeding areas to the voxels of interest at a certain time. A blood pool refers to an amount of blood in a region. A blood feeding area refers to arteries, veins, and the like, which enable blood transport. A voxel refers to a volumetric pixel, which is effectively a three-dimensional (3D) pixel represented, for example, as a cube in 3D space.

Currently, most medical practitioners and researchers select AIF areas manually, by visual inspection of the dynamic evaluation curves in the regions containing the blood pool. However, the manual selection process requires specially trained operations and the results may vary with observers. Moreover, the complicated structures in some tissues—such as brain—can make the detection of the AIF areas difficult due to the scattered distribution of arteries. In addition, manual selection of a global AIF in 3D can be even harder because practitioners and researchers have to select the AIF in each single slice and then combine the selections together. This process can easily lose consistency across the entire 3D volume as well as causing a large effort and cost of time and labor.

Accordingly, an automated AIF determination would be helpful in assessing results of a perfusion study.

### BRIEF SUMMARY

Embodiments of the invention provide tools and techniques for automated arterial input function (AIF) selection used in producing parametric perfusion maps displayed for assisting diagnosis of physiological changes of a patient.

According to one aspect, any imaging modality providing perfusion imaging data containing characteristic parameters associated with a dynamic evaluation curve can be used.

According to an embodiment, a dynamic evaluation curve for each pixel in each slice of imaging data is produced to extract characteristic parameters. The characteristic parameters can include time to peak, maximum slope, and maximum enhancement. In some embodiments, the characteristic parameters being extracted can further include wash-out slope and time to wash-out. Based on the extracted parameters (e.g., time to peak, maximum slope, maximum enhancement, and, optionally, wash-out slope and time to wash-out), pattern recognition and classification can be carried out.

The pattern recognition can include generating two-dimensional (2D) plots based on the extracted parameters. The 2D plots can include a plot of maximum slope vs. time to peak (S vs. T); maximum enhancement vs. time to peak (E vs. T); and, optionally, wash-out slope vs. time to wash-out (W vs. T). For classification, a peak and valley determination can be made with respect to the 2D plots. The data points related to the peaks and valleys can then be used to select the pixels indicating AIF areas.

In one embodiment, the pixels can be selected as indicating AIF areas if the maximum enhancement is greater than the mean enhancement at a point of a peak in a phase of interest on the E vs. T curve; and the maximum slope is greater than the mean slope at a point of a peak in a phase of interest on S vs. T curve; and, when included as part of the characteristic parameters, a wash-out slope is greater than a mean wash-out slope at a point of a peak on the W vs. T curve; and a time to peak is within the peaks on the E vs. T curve and the S vs. T curve; and a time to wash-out is within the peak on the W vs. T curve.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 shows a process flow for perfusion analysis in which an AIF selector according to an embodiment of the invention can operate.

FIGS. 2A-2C show example dynamic evaluation curves for PET (FIG. 2A), MRI (FIG. 2B), and CT (FIG. 2C).

FIG. 3 shows a process flow diagram of a method of selecting AIF areas according to an embodiment of the invention.

FIGS. 4A and 4B show an example time-attenuation curve for a CT study, indicating extraction of characteristic parameters.

FIGS. 5A-5C show detailed process flow diagrams of an example method of selecting AIF areas.

FIG. 6 shows an example AIF selection using parameters extracted from imaging data.

FIGS. 7A and 7B show the difference between the CT-TAC for AIF areas and the surrounding tissues for two example cases.

FIGS. 8A and 8B show an example S vs. T curve and E vs. T curve, respectively.

FIG. 9A illustrates an example of the refined potential peaks selected through the peak validator and the potential valleys determined by the upward zero-crossing method.

FIG. 9B illustrates an example of the real peaks and real valleys selected through the peaks and valleys determiner.

FIG. 10 shows an example computing system for a perfusion analysis system in which embodiments of the invention may be carried out.

FIGS. 11A and 11B respectively show a 2D plot of S v. T and E v. T for a before-infarcted study of an experiment.

FIGS. 11C and 11D respectively show a 2D plot of S v. T and E v. T for an after-infarcted study of an experiment.

FIGS. 12A and 12B respectively illustrate the automated selection of potential peaks and valleys (FIG. 12A) and the real peaks and valleys (FIG. 12B) for the before-infarcted study of an experiment.

FIGS. 12C and 12D respectively illustrate the automated selection of potential peaks and valleys (FIG. 12C) and the real peaks and valleys (FIG. 12D) for the after-infarcted study of an experiment.

FIGS. 13A and 13B show binary images of the results of the automated detection of AIF pixels for the before-infarcted study and after-infarcted study, respectively.

FIGS. 14A and 14B show the average TACs of selected AIF pixels for the before-infarcted study and after-infarcted study, respectively.

FIGS. 15A and 15B show example original anatomical images for the before-infarcted study and the after-infarcted study, respectively.

FIGS. 16A and 16B show perfusion maps for the before-infarcted study and the after-infarcted study, respectively.

FIGS. 16C and 16D show 3D perfusion volumes for the before-infarcted study and the after-infarcted study, respectively.

FIGS. 17A-17C respectively show a 2D plot of S vs. T, E vs. T, and W vs. T for an abdominal perfusion study experiment.

FIGS. 18A-18B show an example of the automated process on an S vs. T curve.

FIGS. 19A-19B show an example of the automated process on an E vs. T curve.

FIGS. 20A-20B show an example of the automated process for a W vs. T curve.

FIG. 21 shows a 3D AIF region of an artery resulting from the automated process of the example.

FIG. 22 shows an average PET-TAC for pixels in an AIF region.

FIGS. 23A and 23B show perfusion maps of the kidneys and upper GI.

FIGS. 24A and 24B show the fused perfusion maps with CT anatomy images.

FIG. 25 shows a 3D perfusion volume.

### DETAILED DISCLOSURE

Embodiments of the invention provide tools and techniques for automated arterial input function (AIF) selection used in producing parametric perfusion maps displayed for assisting diagnosis of physiological changes of a patient.

Tissue perfusion can be a measure of capability of central cardiovascular mechanisms to deliver oxygen to peripheral tissue for meeting metabolic needs. Since perfusion is closely related to oxygen and nutrient transfer, analysis of perfusion and associated parameters can be used for diagnosis of physiological changes, such as ischemic stroke, tumor, cardiac infarction and inflammation.

In a perfusion study, perfusion quantification may be carried out by determining a concentration of tracer inside a tissue. The AIF is one of the functions, which may also include a consideration of transport (distribution of transit time over an individual voxel) and residue (fraction of injected tracer remaining in the tissue voxel of interest at a moment (t) in time following an ideal bolus injection), used to define a concentration of tracer inside a tissue.

In order to increase accuracy and efficiency of a process for determining AIF areas for perfusion analysis, and to reduce variability among clinicians analyzing perfusion, various embodiments of the invention provide systems and methods for automatically determining AIF areas from perfusion imaging. The automated AIF determination of embodiments of the invention is applicable to many imaging modalities, such as CT, PET, single photon emission computed tomography (SPECT), ultrasound, luminescent, fluorescent, and MRI, as well as being applicable across many types of tissue.

Embodiments provide an automated determination of arterial input function, which can then be used to generate and analyze parametric perfusion maps.

By automating the process of finding the AIF, time and labor consumption can be reduced and, importantly, the inherent inter-operator variability and inconsistency in parallel experiments or when comparing changes in follow-up studies during treatment therapy can be removed. In addition, the automated determination of AIF areas of an entire 3D volume can be executed at one time as opposed to manual determination of AIF areas which can only be executed for one slice. Moreover, because the automated determination of AIF areas is based on a pixel-wise characteristics analysis, an accurate and effective determination is possible even for blood supply areas with scattered distribution.

A general process for presenting data obtained from a perfusion study involves taking data obtained from imaging a tracer injected into a patient and presenting the dynamic information as a parametric image associated with anatomy. As previously described, selecting the AIF areas is an important step in obtaining quantitative measurements of blood flow through a region of interest.

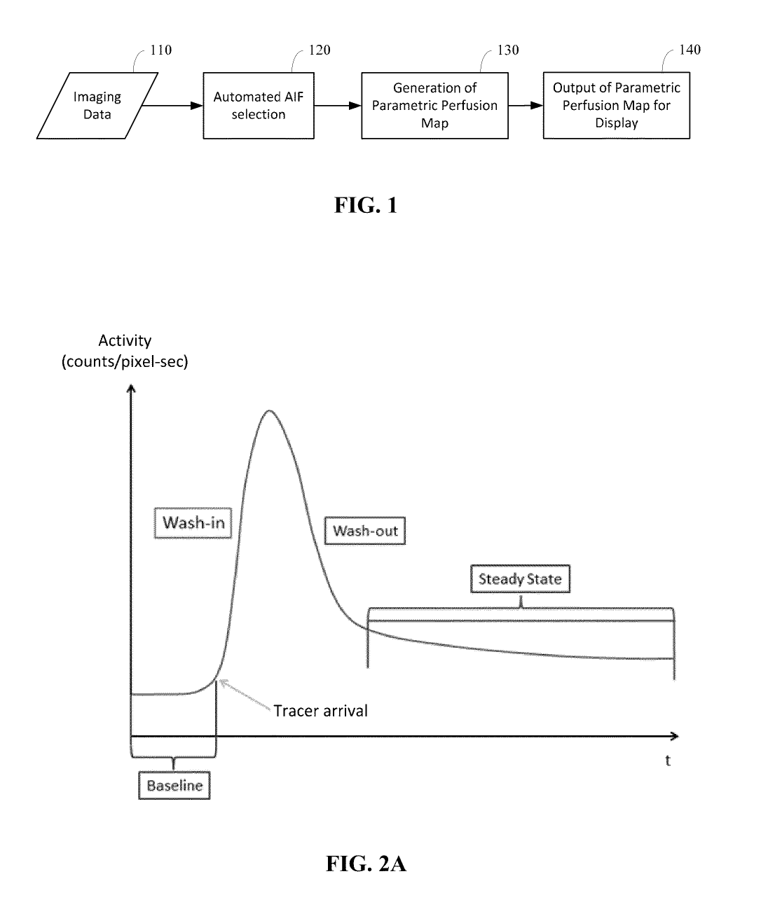

FIG. 1 shows a process flow for perfusion analysis in which an AIF selector according to an embodiment of the invention can operate. Referring to FIG. 1, imaging data 110 from an imaging modality such as MRI, CT, or PET can be input to an automated AIF selection module 120 for selection of the AIF areas. The automated AIF selection (120) can be carried out from within a software application used for displaying a 3D or 2D rendering of the imaging data 110. The software application may be a stand-alone application or an application associated with a particular imaging apparatus. Once the selection of the AIF is obtained (120), a parametric perfusion map can be generated (130) and the map output for display (140).

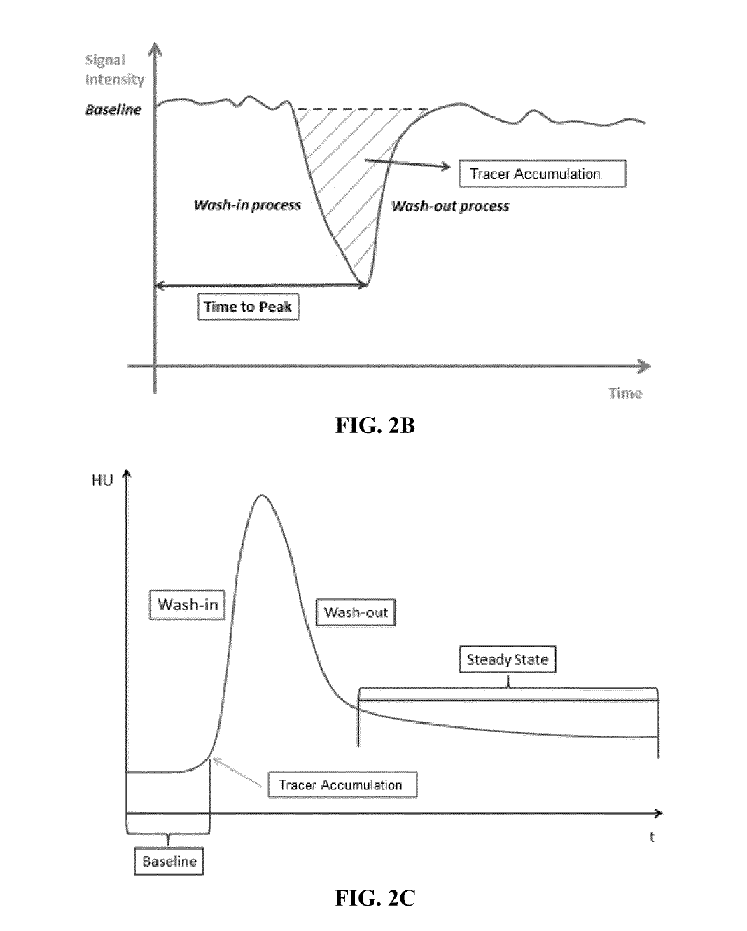

The imaging data 110 from which the AIF areas are selected can include data associated with producing dynamic evaluation curves (e.g., from tracer enhancement curves). FIGS. 2A-2C show example dynamic evaluation curves for PET (FIG. 2A), MRI (FIG. 2B), and CT (FIG. 2C). The various stages of perfusion are labeled, including baseline, tracer wash-in, tracer wash-out, and steady state.

Referring to FIG. 2A, the kinetics of the tracer, as shown by the dynamic evaluation curve (e.g., the PET time activity curve), represents the first pass of the tracer travelling through the tissues. Since the signal intensities present the amount of the tracers in the corresponding pixels, the pixel intensity reflects the blood flow and distribution in that area. The radioactivity changing around a tissue over time generates the tracer enhancement curves for the tissue.

Referring to FIG. 2B, the MRI perfusion study tracks bolus (e.g., the tracer used for the MRI perfusion study) through dynamic susceptibility contrast (DSC-MRI). The pixel intensities from the MRI present signal intensities (without needing to transfer into an activity evaluation as performed for PET). A time-intensity curve such as shown in FIG. 2B can be obtained for each pixel through the dynamic evaluation of sequential images to present the tracer kinetics. Abnormal parts of tissue tend to show less signal loss compared to surrounding tissues in time-intensity curves.

Referring to FIG. 2C, the kinetics of the tracer (e.g., contrast bolus), representing a first pass of the tracer traversing through the tissue microvasculature, describes how the X-ray attenuation of a CT scan changes over time. The areas with normal perfusion uptake higher contrast and present brighter images than the ischemic areas with reduced perfusion. In dynamic CT imaging, sequential images are obtained over a defined period of time to trace the kinetics of contrast bolus in the blood pool and tissues. The principle is similar to that of DSC-MRI. The Hounsfield units (HU) changing over time allows the creation of the enhancement curves, referred to as time-attenuation curves, for the tissue, region of interest or individual pixels.

As can be seen from FIGS. 2A-2C, the dynamic evaluation curves indicate similar characteristics—with peaks during tracer accumulation (wash-in) and wash-out, followed by a steady-state. According to certain embodiments of the invention, the AIF can be automatically selected by classifying the characteristic parameters of the image pixel's dynamic evaluation curves between the blood pool and tissues.

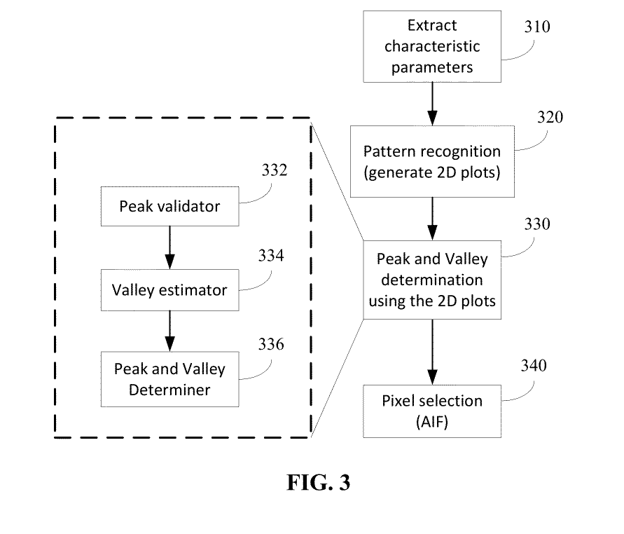

FIG. 3 shows a process flow diagram of a method of selecting AIF areas according to an embodiment of the invention. Referring to FIG. 3, imaging data (such as imaging data 110 of FIG. 1) can be received and may undergo an optional pre-processing step (not shown). The imaging data contains information of position (slice number), time point (in a time series), and pixel ordinates (e.g., x and y positions). The pre-processing step may be any suitable filtering or processing of data received from an imaging modality, for example, de-noising smoothing techniques or curve-fitting techniques may be applied.

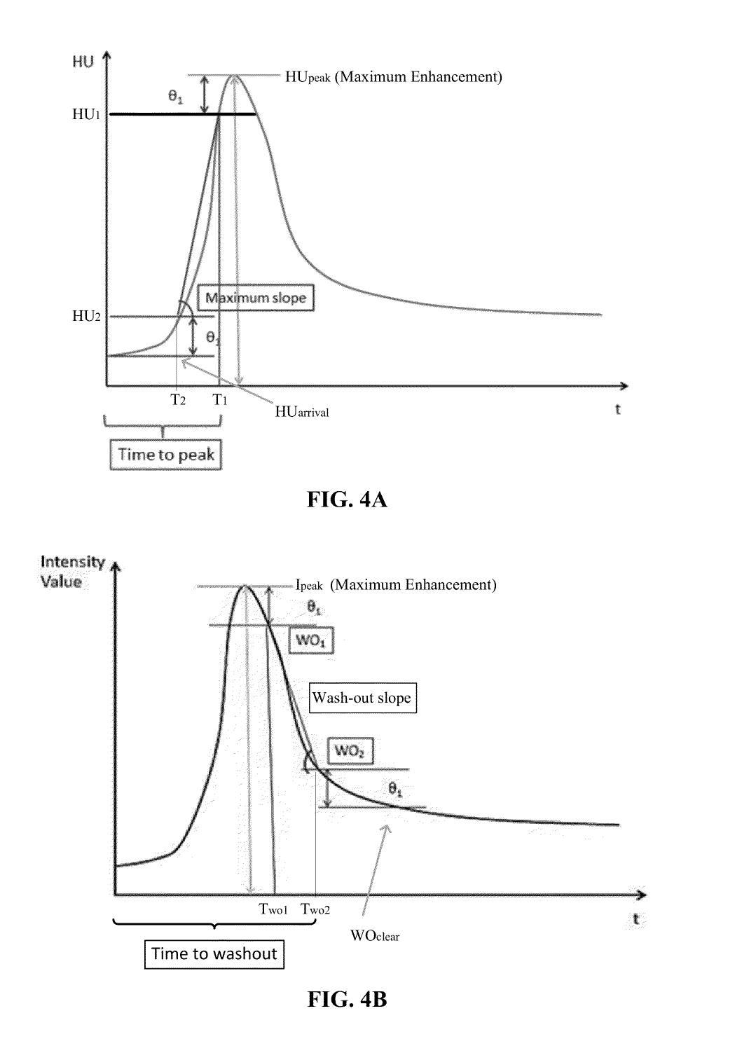

A dynamic evaluation curve for each pixel in each slice is produced to extract the desired characteristic parameters (310). The characteristic parameters can include the three parameters of time to peak, maximum slope, and maximum enhancement, such as described with respect to FIG. 4A. In another embodiment, the characteristic parameters can include the three parameters of maximum enhancement, wash-out slope, and time to wash-out, such as described with respect to FIG. 4B. In certain embodiments, both the characteristic parameters such as described with respect to FIG. 4A and the characteristic parameters such as described with respect to FIG. 4B are used.

The characteristic parameters are extracted from acquired imaging data to determine perfusion information about a subject. Based on the extracted parameters (e.g., time to peak, maximum slope, maximum enhancement, and, optionally, wash-out slope and time to wash-out), pattern recognition can be carried out (320).

The pattern recognition can be carried out to identify relationships between extracted characteristic parameters. The identified relationships can be used to classify datapoints of the imaging data for automatic tissue segmentation and AIF area determination.

The pattern recognition can include generating two-dimensional (2D) plots based on the extracted parameters. The 2D plots can include a plot of maximum slope vs. time to peak (S vs. T); maximum enhancement vs. time to peak (E vs. T); and, optionally, wash-out slope vs. time to wash-out (W vs. T).

A Peak and Valley determination (330) can be made with respect to the 2D plots. For example, the data can be processed by a peak validator 332 to obtain potential peaks in the data curve of the 2D plots and a valley estimator 334 to obtain potential valleys in the data curve of the 2D plots. The potential peaks and valleys are then used to determine the real peaks and valleys (as opposed to peaks and/or valleys associated with noise or other artifacts) in the peak and valley detector 336. The resulting dynamic curve data is used to select the pixels indicating AIF areas (340).

The pixels in the AIF areas are the ones with maximum enhancements greater than the mean enhancement at the point of the peak in the AIF phase, with the condition that the maximum slopes are bigger than the mean slope at the same point. The process shown in FIG. 3 may be carried out in an automated AIF selection module (such as module 120 of FIG. 1) of a perfusion analysis system.

It should be understood that while embodiments are described herein as generating 2D plots from which features are extracted for use in selecting pixels corresponding to AIF areas, other methods of representing the data related to tracer behavior (including wash-in and wash-out) are contemplated. For example, pattern recognition and classification may be carried out through numerical analysis without generating the plots and applying a peak and valley determination.

FIGS. 4A and 4B show an example time-attenuation curve for a CT study, indicating extraction of characteristic parameters. As shown in FIG. 4A, time to peak, maximum slope, and maximum enhancement can be extracted for each pixel of the 4D imaging perfusion data. As shown in FIG. 4B, wash-out slope and time to wash-out can also be extracted for each pixel of the 4D imaging perfusion data.

As illustrated in FIG. 4A, HUₐᵣᵣᵢᵥₐₗ is the value of the change in attenuation (e.g., Hounsfield Unit) at the point of arrival of the tracer (or “bolus”) and HUₚₑₐₖ is value of change in attenuation at point of the maximum enhancement.

To address system noise that may exist in a CT system, the calculations using CT imaging perfusion data can include thresholds such as a peak-dependent threshold θ₁ to minimize negative affects to the determination of the maximum slope of a CT-TAC. Thus, the maximum slope can be given as:

$$\frac{{HU}_{1} - {HU}_{2}}{{T}_{1} - {T}_{2}} , \\ where$$

$${HU}_{1} = {HU}_{peak} - {θ}_{1}$$

$${HU}_{2} = {HU}_{arrival} + {θ}_{1}$$

$${θ}_{1} = α * {HU}_{peak} , \\ 0 < α < 1.$$

An optimal value of a can be determined by selecting a steady and characteristic upslope. HU₁ and HU₂ are values of changes in attenuation at the two points selected by threshold θ₁, and T₁ and T₂ are the corresponding time slices.

The time-to-peak is the time at which a change in attenuation reaches the second point selected by the threshold θ₁ that is temporally closer to the maximum enhancement (e.g., at HUₚₑₐₖ).

If a pixel's time-attenuation curve does not show any of the three characteristic parameters of time to peak, maximum slope, and maximum enhancement, then the pixel can be ignored as being either too noisy for calculation or as being in background of image (and not containing useful information).

Referring to FIG. 4B, the wash-out parameter can be calculated in a manner similar to the determination of maximum slope, but on the side corresponding to the tracer being cleared (e.g., the wash-out process). For example, the calculations can use the peak-dependent threshold θ₁ to minimize negative affects to the determination of the wash-out slope of the CT-TAC by using the peak-dependent threshold θ₁ to select two points, the gradient of which is an estimation of the wash-out slope. In particular, the gradient (e.g., the wash-out slope) is given as:

$${WO}_{slope} = \frac{\vert {WO}_{2} - {WO}_{1} \vert}{{T}_{wo 1} - {T}_{wo 2}} , \\ where$$

$${WO}_{1} = {I}_{peak} - {θ}_{1}$$

$${WO}_{2} = {WO}_{clear} + {θ}_{1} .$$

Iₚₑₐₖ is the intensity value (or the associated unit for the particular imaging modality) at the point of maximum enhancement and WO_{clear} is the value at the point where the tracer is cleared up (this value may represent where the tracer is completely cleared up). WO₁ and WO₂ are the values at the two points selected by threshold θ₁, and T_{wo1} and T_{wo2} are the corresponding time slices (e.g., the time values in the acquisition time serial). Time to wash-out can be the time when the dynamic evaluation curve reaches WO₂.

As described above, through automated identification and calculation processes, characteristic parameters including maximum enhancement, maximum slope and time-to-peak can be extracted from a dynamic evaluation curve. It should be understood that although a CT-TAC is illustrated in this example, embodiments are not limited to extracting these three characteristics from CT imaging data. Rather, any imaging data having related activity with peaks and valleys can be used to extract the three characteristics. For example, the MRI and PET dynamic evaluation curves shown in FIGS. 2A and 2B can undergo analogous extraction (with or without using a peak-dependent threshold or other noise removal technique).

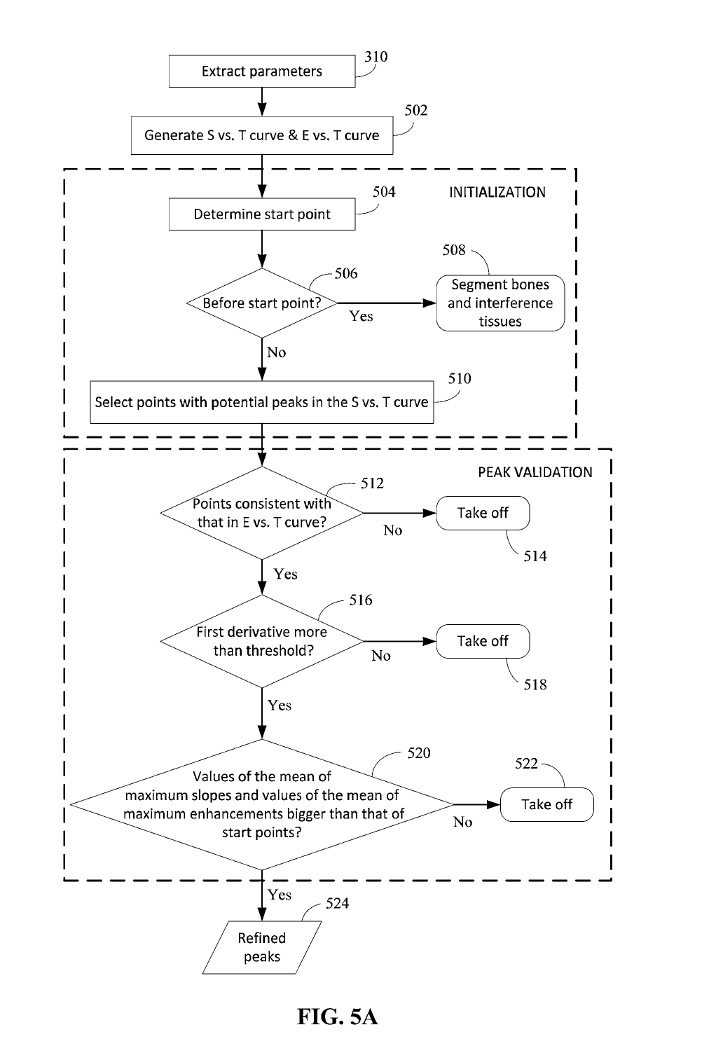

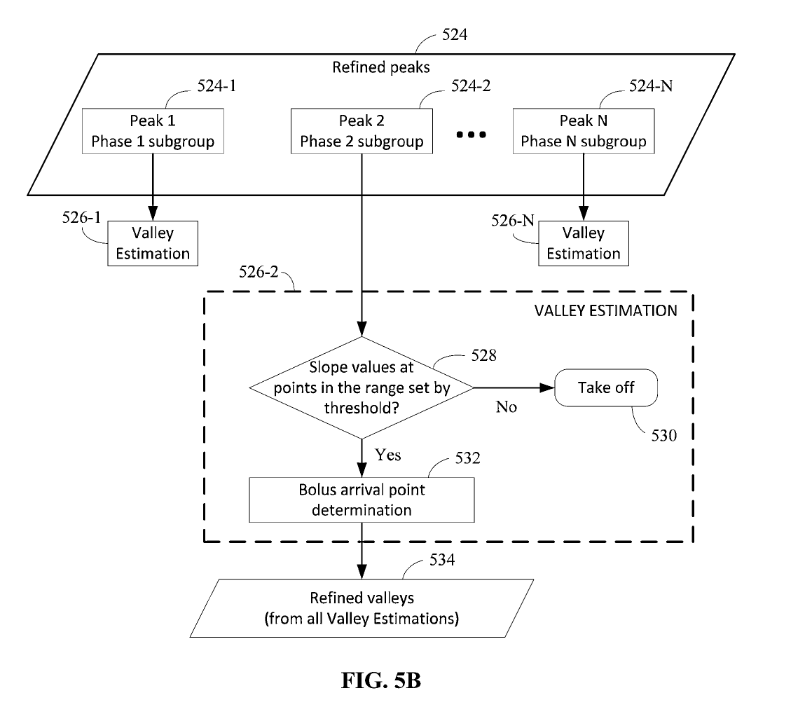

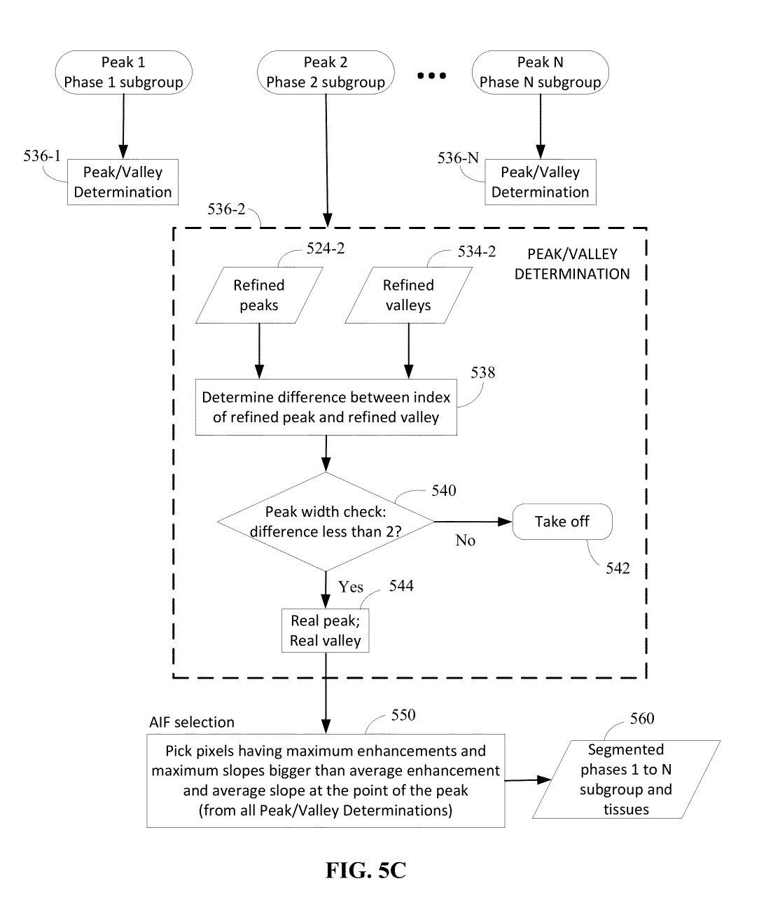

FIGS. 5A-5C show detailed process flow diagrams of an example method of selecting AIF areas. Referring to FIG. 5A, the process can begin with extracting parameters 310 such as described with respect to FIG. 3. Then, when generating the 2D plots, at a minimum, the S vs. T curve and the E vs. T curve are generated (502). An initialization process can be performed to segment pixels indicating bones and interference tissues. For example, a start point can be determined (504) and a determination can be made as to whether a data point is from a time before the start point (506). If the time is before the start point, then bones and interference tissues can be segmented (508). Once the start point begins, points with potential peaks in the 2D plot can be selected (510). According to some embodiments, the S vs. T curve is used as part of the initialization processes; however, embodiments are not limited thereto.

In one embodiment, to segment (i.e., remove) bones and potential interference tissues, a threshold θ₂ can be set to provide an absolute number limit for the first derivative of the S vs. T curve based on the principle that bones and interference tissues show sharp slopes. To find the start point (the bolus arrival point of the first peak), a zero-crossing method can be used. For some imaging modalities, such as CT, the start point is located in the first valley. Therefore, bones and interference tissues can be automatically segmented by setting the time restriction before the start point. This is illustrated in FIG. 7A, which shows the tissue TAC values being less than the AIF TAC values, particularly at a time before the first peak of the AIF TAC.

Returning again to FIG. 5A, from the start point, the zero-crossing method can look for the upward zero-crossing in the first derivative of each point on the S vs. T curves. The potential peak selection (510) also uses the zero-crossing method by looking for downward zero-crossings in the first derivatives of the S vs. T curves. Once the points are selected in the S vs. T curve, peak validation can be carried out.

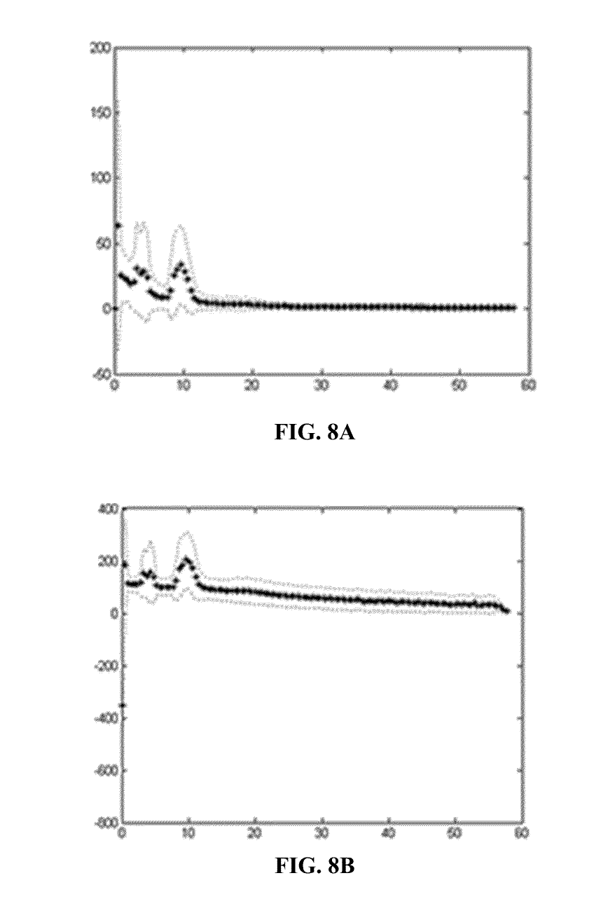

In one embodiment, a determination is made as to whether the selected points indicative of potential peaks in the S vs. T curve are consistent with those in the E vs. T curve (512). FIGS. 8A and 8B show an example S vs. T curve and E vs. T curve, respectively. The top curves in FIGS. 8A and B, respectively, indicate the mean plus standard variation of the slopes and the mean plus standard variation of the enhancements. The lower curves in the FIGS. 8A and 8B respectively, indicate the mean minus standard variation of the slopes and the mean minus standard variation of the enhancements.

As can be seen in the example of FIGS. 8A and 8B, during the early time before the bolus arrives, there can be sharp peaks or valleys, or both. For CT and similarly fast acquisition time modalities, sharp peaks or valleys can be caused by large attenuations due to bones and interference tissues having fluid (not blood) inside. Whereas, after the tracer arrives, a regular pattern can occur as shown: the areas containing blood pool present a parabola, gradually ascending and then descending on both S vs. T and E. vs. T curves. In imaging modalities having a longer acquisition time, such as PET, the pattern may be sharper (due to rapid transition between peaks), and interfering tissues and/or bones may indicate according to the expected patterns for that imaging modality.

The peaks in FIGS. 8A and 8B appear to occur at nearly the same time points (on the axis of time to peak). The number of the parabolas, referred to herein as “phases”, varies with tissues due to the variable physiological processes in different tissues. The number can also change based on the scan phases we are imaging. For example, in the heart, if both the right and left ventricles are imaged, there might be two or three peaks: the blood pool in right ventricle, followed by the blood pool in left ventricle and perhaps right ventricle recirculation. Whether there is recirculation or not depends on the amount of tracer infused. In the liver, there might be two peaks: arterial phase and venous phase. Therefore, the emergence of different numbers of phases relies on the tissues and imaging protocol. Because the variables are known before a perfusion study, the particular pattern can be known.

Returning again to FIG. 5A, if a point is not consistent between the S vs. T curve and the E vs. T curve, then the point is removed from being indicated as a peak (514). If the point indicative of a potential peak in the S vs. T curve is considered consistent with that in the E vs. T curve, then a determination is made as to whether the first derivative of the curve at the point is more than a threshold (516). This threshold (θ₃) can be provided to remove small peaks (which may be indicative of noise or other signals).

For cases similar to the example described with respect to FIGS. 8A and 8B, to remove very small recirculation peaks that can be neglected (because it can be assumed that wash-out has occurred), the values of the mean of maximum slopes and the values of the mean of maximum enhancements should be bigger than those at the start points, respectively. Accordingly, a determination can be made whether the mean values at the points are bigger than that of the start points (520). If the values are not bigger, then the point can be removed (522).

Results of peak validation, for example as described with respect to steps 512-522, can provide data regarding the refined peaks 524.

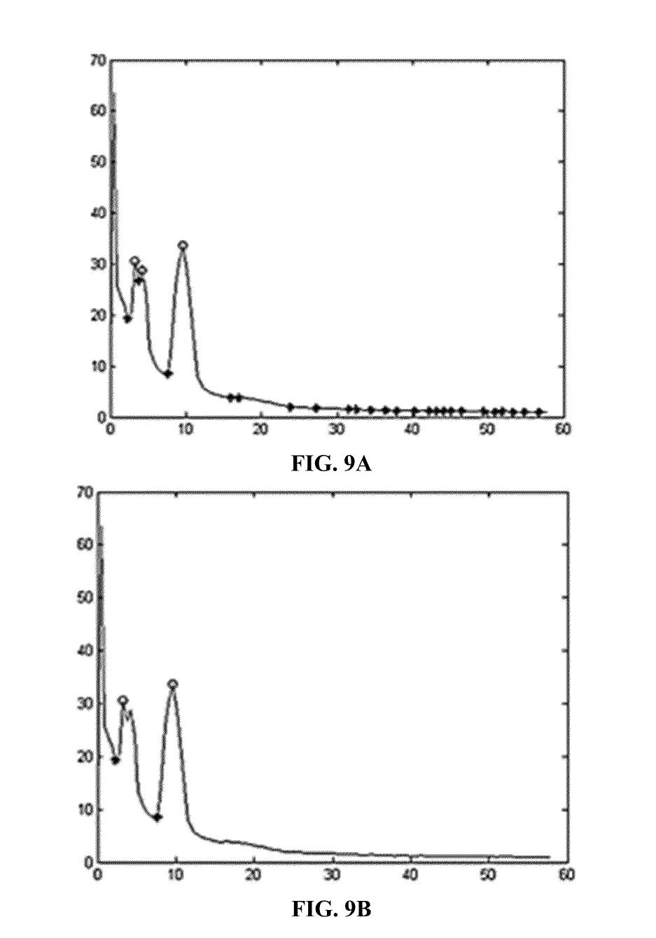

FIG. 9A illustrates the refined potential peaks—peak candidates—selected through the peak validator and the potential valleys determined by the upward zero-crossing method (see marked data points). FIG. 9B shows the real peaks and real valleys selected through the peaks and valleys determiner (four marked points remain).

Referring to FIG. 5B, a subgroup can be assigned for each peak candidate (e.g., 524-1, 524-2, . . . , 534-N) in the data regarding the refined peaks 524. A subgroup can contain all the potential valleys having time to peaks between that of the peak (with which the subgroup is assigned) and that of the previous peak. For example, the peak candidate Peak 1 can have a phase 1 subgroup assigned that contains a collection of points of potential valleys between the start point and the first peak (with the start point included). The peak candidate Peak 2 can have a phase 2 subgroup assigned that contains a collection of points of potential valleys between the first peak and the second peak. This arrangement can continue for all peak candidates through Peak N, which is assigned a phase N subgroup containing a collection of points of potential valleys between the previous peak (e.g., N−1) and its peak.

Valley estimation can then be carried out using the refined peak data. Since the bolus arrival point for each phase is generally among the few lowest valleys in each subgroup, a peak-dependent threshold θ₄ may be used to obtain the valley range. For example, a determination can be made as to whether the slope values are within the range set by the threshold (528).

The arrival time point for each peak candidate is the last valley within the valley range assigned to the phase. The threshold θ₄ can be used to remove small peaks that should be neglected.

In certain embodiments, the threshold θ₄ and valley range R_{valley} can be given as:

θ₄=β*Sₚₑₐₖ

R_{valley}=S_{lowest}+θ₄

Sₚₑₐₖ is the mean of the maximum slopes in the range containing the peak point, S_{lowest} is the lowest mean of the maximum slopes among all the boxes in this subgroup, and β is a variable for setting the threshold. An optimal determination of the threshold θ₄ is to ensure that the valley range will not cover the points in the upgrade part of the S vs. T curve, and at the same time, to remove the small and noisy valleys.

If the slope value is not within the range R_{valley}, the point can be removed (530); however, if the slope value is within the range, a determination of the bolus arrival point can be made (532) and the results of the valley estimations for each subgroup can provide the data regarding the refined valleys 534.

FIG. 5C illustrates peak/valley determination using, for example, a peak and valley determiner 336 such as shown in FIG. 3. Referring to FIG. 5C, the refined peaks and refined valleys obtained through the peak validator and valleys estimator can be used to determine the phases having real peaks and valleys. Each phase subgroup can have its associated peaks and valleys determined (536-1, 536-2, . . . , 536-N). For example, the difference between the index of a refined peak and refined valley (538) can be determined using the refined peaks 534-2 and refined valleys 534-2 for the phase 2 subgroup. The “index” refers to the coordinates of the points on the plots. A peak width threshold can be used to ensure that the peak and the valley are not nearby each other. For example, a width threshold may be 2 time segments. A determination can then be made whether the index difference (538) is less than 2 (540). If the peak width is too large, the point can be removed (542). If the peak width is within the threshold, then the point can be determined to be a real peak or a real valley (544).

By using the automatically determined phases with detected peaks and valleys and on the basis of the physiological condition of the tissue, the phases containing AIF can be selected (550). Since the general tissue perfusion is also present in the AIF phase, the AIF can be determined by performing calculations refining the blood pool. The maximum enhancement and the maximum slope of an AIF are generally higher than that of tissues, and these two variables depend on the amount of tracer and the injection rate. In the general situation, the pixels that are in the AIF areas are the ones with the maximum enhancements bigger than the mean enhancement at the point of the peak in the AIF phase, with the condition that the maximum slopes are bigger than the mean slope at the same point.

Accordingly, AIF selection (550) can be carried out by picking pixels having maximum enhancements and maximum shapes bigger than the average enhancement and average slope at the point of the peak. The results of AIF selection provide segmented tissues (560).

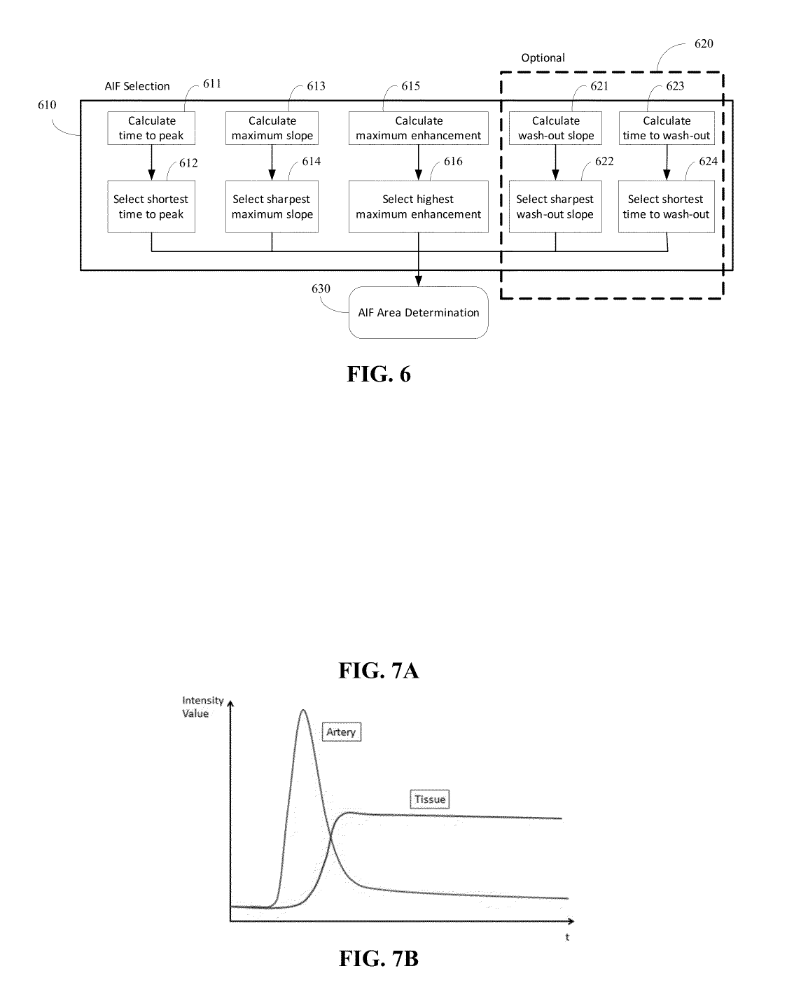

FIG. 6 shows an example AIF selection using parameters extracted from imaging data. The AIF selection shown in FIG. 6 may be carried out as part of step 550 of FIG. 5C. Referring to FIG. 6, AIF selection can be carried out by calculating time to peak 611 and selecting shortest time to peak 612; calculating maximum slope 613 and selecting sharpest maximum slope 614; and calculating maximum enhancement 615 and selecting highest maximum enhancement 616. In certain embodiments, additional computations 620 can be carried out. For example, the AIF selection 610 can further include calculating wash-out slope 621 and selecting the sharpest wash-out slope 622 and calculating time to wash-out 623 and selecting the shortest time to wash-out 624. The additional computations 620 can be optional, depending on the type of tracer and the size of blood feeding areas undergoing the perfusion studies. The optional computations 620 can be included when trappable tracers are being used for the perfusion studies.

For example, when blood feeding areas are large, such as the area of the left ventricle and arteries (as compared to myocardium—which also does not indicate a high uptake of tracer), the difference between blood pool areas and tissues with respect to maximum slope and maximum enhancement stands out.

In contrast, when blood feeding areas are very small, especially when the uptake of the tracer in some tissues, such as kidney, is too large to provide a clearly distinguishable difference between arteries and such tissues, it can be difficult to determine the appropriate maximum slope and the maximum enhancement. A non-diffusible tracer may be biologically trapped by certain tissues. Thus, inside blood pool areas, a non-diffusible tracer behaves similarly to a diffusible tracer. However, for the certain tissues, the non-diffusible tracer can become completely trapped by the tissue. In such cases, the tracer cannot be washed out from the tissue. This can be seen in FIG. 7B where a tissue traps the tracers. It should be noted that the maximum enhancements of tissues are not necessarily lower than those of arteries.

Examples of such studies include cerebral perfusion analysis and abdominal perfusion analysis. For these cases, characteristic features which are more distinguishable than the maximum slope and the maximum enhancement are extracted to execute the pattern recognition and determine the appropriate AIF areas. For example, the optional computations 620 can be performed. For embodiments incorporating wash-out parameter extraction, the peak validation can be carried out in a similar manner as with the S vs. T and E vs. T curves. For example, zero-crossings in the first derivative that exceed a threshold are searched. Valley estimation may be omitted for the W vs. T curve because the W vs. T curve tends to have a single peak. Segmentation readily achievable because of the differences in diffusion of the tracer from the tissue.

Pixels having the selected characteristics (with or without the optional features from 620) can be used to provide an AIF area determination 630.

An automated AIF selector is presented that is applicable to many imaging modalities and tissue types with slight variations according to the physics of an imaging modality and tracer properties. For example, completely trappable tracers will not cause recirculation. In addition, PET imaging is different from CT imaging in that there is less interference from bones and fluid.

In particular, unlike the CT imaging data affected by the bones and fluids resulting in large attenuation, the start point (the tracer arrival point of the first peak) in the PET imaging data does not appear like a valley, but simply is an initial position for the following peak. This point can be obtained by looking for the first derivative that exceeds a threshold. Peaks and valleys are easier to be picked by simply looking for downward (upward) zero-crossings in the first derivative that exceed another threshold.

The number of the peaks in the S vs. T curve varies with the physiological conditions of different organs.

Therefore, in some of such cases, the automated determination of peaks and valleys can be simplified to omit steps for the noise removal. According to an embodiment, the automated determination can be carried by using the zero-crossing method and applied thresholds for the S vs. T, E vs. T, and W vs. T curves.

According to an exemplary embodiment of present invention, the automated determination of AIF areas of present invention can be applied to perfusion analysis of any tissues with slight adjustment, because automated determination of AIF areas is not only based on analysis of mathematical characteristics of time-attenuation curves associated with the AIF areas but also based on analysis of physiological process of different tissues.

According to an exemplary embodiment of present invention, since pixel-wise dynamic evaluation curves generated by various perfusion imaging modalities, such as time-attenuation curves generated by CT, time-intensity curves generated by MRI, and time-activity curves generated by PET, have similar characteristics, automated determination of AIF areas can be carried out using the approaches described herein.

A greater understanding of the present invention and of its many advantages may be had from the following example, given by way of illustration. The following example is illustrative of some of the systems, methods, applications, embodiments and variants of the present invention. They are, of course, not to be considered in any way limitative of the invention. Numerous changes and modifications can be made with respect to the invention.

### Example

### Computing System

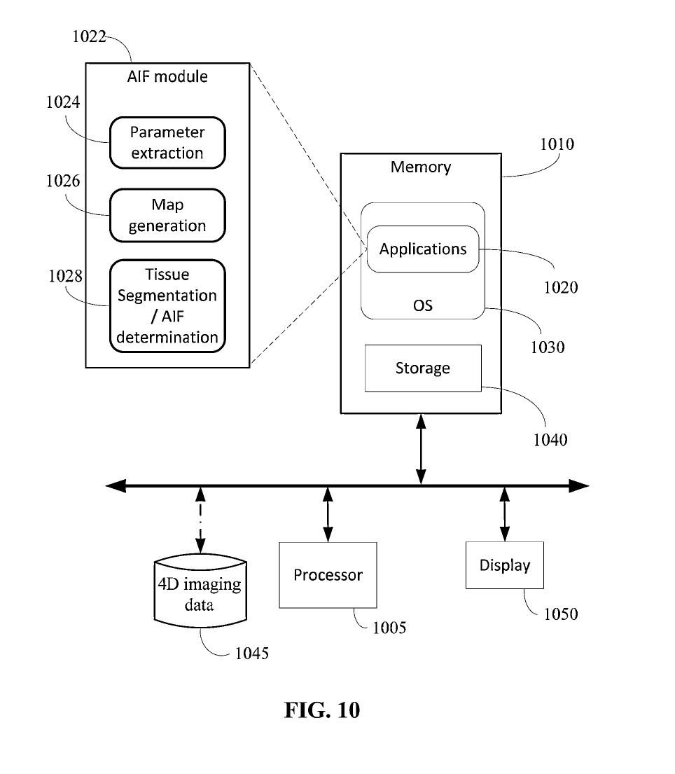

FIG. 10 shows an example computing system for a perfusion analysis system in which embodiments of the invention may be carried out.

According to an embodiment, the system can include a processor 1005 and memory 1010 in which one or more applications 1020 may be loaded. The processor 1005 processes data according to instructions of the applications 1020.

The applications 1020 can include an AIF module providing instructions for performing automated AIF selection as described herein. The AIF module 1020 can include parameter extraction 1024, map generation 1026, and tissue segmentation/AIF determination 1028. The applications 1020 can be run on or associated with an operating system 1030 that can also be loaded into the memory 1010. Other applications may be loaded into memory 1010 and run on the computing device, including various client and server applications. Non-volatile storage 1040 may be available within memory 1010 to store persistent information that should not be lost if the system is powered down. A database 1045 storing 4D imaging data can be coupled to the system via wired or wireless connections.

Visual output can be provided via a display 1050. Input/Output (I/O) devices (not shown) such as a keyboard, mouse, network card or other I/O device may also be included. It should be understood the any computing device implementing the described system may have additional features or functionality and is not limited to the configurations described herein.

### Example

### Myocardial Perfusion Studies

An example myocardial perfusion study is carried out illustrating the use of a CT perfusion study using an embodiment of an automated AIF selection as described herein.

To assess myocardial perfusion, the region of interest (ROI) that is selected as AIF areas for perfusion calculation is generally set either on the aorta or on the left ventricle. However, AIF areas should be positioned in all the areas that feed blood into the tissues of interest rather than only the aorta or left ventricle.

Generally, the circulatory system in the body can be divided into either pulmonary circulation or systemic circulation. Deoxygenated blood returns from the body through the systemic venous system into the two major veins, the cranial and the caudal vena cava, which terminates in the right atrium. From the right atrium the deoxygenated blood is pumped to the right ventricle and subsequently into the main pulmonary artery. The main pulmonary artery quickly bifurcates into the right and left pulmonary arteries, which supply their respective lungs. Blood subsequently passes through the pulmonary capillaries where gas exchange occurs and continues into the pulmonary veins, left atrium, left ventricle and aorta.

The coronary arteries supply the myocardium—the heart muscle—and originate at the proximal part of the aorta. The major arteries of the coronary circulation are the left coronary artery, which divides into left anterior descending and circumflex branches, and the right coronary artery. Both arteries originate at the base of the aorta and lie on the surface of the heart. These arteries may also be referred to as the epicardial coronary vessels. These arteries also distribute blood flow to different regions of the myocardium and are classified as heart “end circulation” because they are the only blood supply source for the myocardium. Coronary artery disease is caused by the blocked coronary arteries, and the damage of any of these three arteries may lead to critical outcomes.

Based on the above described system, the pulmonary veins, left atrium, left ventricle, aorta and the arterioles (the last small branch of the arterial system from where the blood is released into the capillaries) are considered AIF areas.

### Animal Preparation

In this example experiment, an ovine weighing 50 kg was used as a model of myocardial ischemia and reperfusion in this study after approval from Institutional Animal Use and Use Committee (IACUC). Myocardial infarction is induced by using cardiac catheterization to occlude the blood flow of left anterior descending (LAD) coronary artery for 90 minutes. CT scans were performed prior to and after the intervention.

### CT Scan Imaging Protocol

The CT scan was performed with a 128-slice CT multi-row detector CT (MDCT) scanner (Biograph mCT, Siemens, Knoxville, USA) with a gantry rotation time of 300 ms. For the tracer, a contrast bolus of iodine (Omnipaque 350) was infused through the vein at a rate of 4 ml/s. In the before-infarcted study, the amount of contrast bolus use was 12 ml and after-infarcted study, the amount was 24 ml. The difference in tracer amount is to test the tracer-dependency of the automatic AIF selection algorithm. For both of the studies, a saline chaser of 64 ml at the same injection rate as that of contrast bolus was utilized for wash-out process. The scan was started 2 s after the initiation of the tracer injection and continued for 70 s such that the tracer can move through the entire heart. 24 slices of images were obtained with 3 mm slice thickness. The image protocol was performed at 80 KV due to the photoelectric effect for 80 KV photons, which are closer to the “k-edge” of iodine. Based on this kilovolt, the constant milliampere-second is set to be 120 mAs. Values for effective radiation dose were calculated by multiplying the dose-length product with a conversion factor (k=0.014 mSv/mGy×cm).

After imaging, a cardiac phase of 52% was selected for both before-infarcted and after-infarcted studies, to achieve the least motion and artifacts. A medium-smooth convolution kernel (B30f) was chosen to ideally reflect the iodine content in the myocardium. The axial images obtained by cine mode scan were reconstructed into 3600 images and a beam-hardening correction was applied in the reconstruction kernel to remove beam-hardening artifacts that mimics the appearance of myocardial perfusion defects.

### Automated AIF Determination

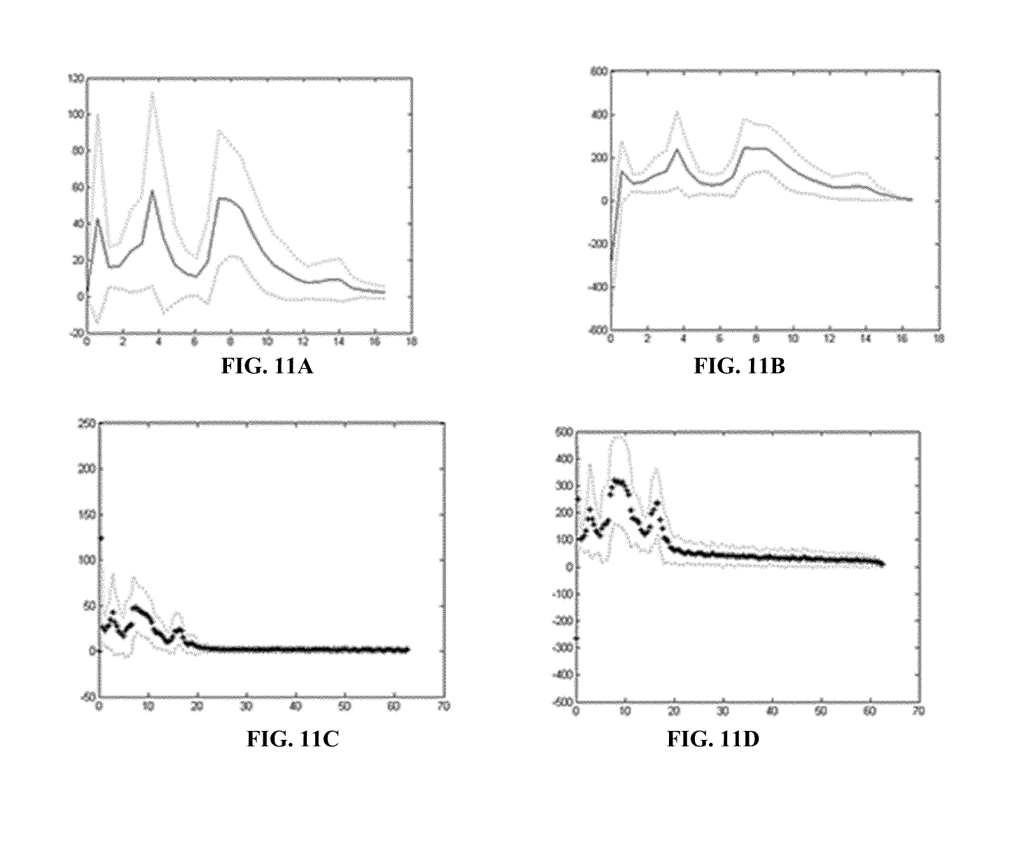

To extract the characteristic parameters and perform the pattern recognition (e.g., steps 310 and 320 of FIG. 3), the threshold constant α was set to 0.3 to provide a steady and characteristic upslope. The three parameters were extracted: maximum enhancement, maximum slope and time-to-peak. FIGS. 11A-11D show the 2D plots (S vs. T curve and E vs. T curve) after converting the 3D parameter maps for the before infarcted study and after infarcted study.

Referring to FIGS. 11A-11D, the very sharp peaks or valleys that occur before the contrast bolus arrives are caused by bones and interference tissues with fluid (not blood) inside. After the contrast bolus arrival, since the contrast bolus was infused from the vein, the first peak (or parabola) represents the tracer enhancement of the right ventricle and coronary arteries. The second peak (or parabola) demonstrates the tracer enhancement of the left ventricle/atrium, pulmonary veins, the aorta and its branches. The third peaks on both curves in the after-infarcted study are associated with the blood recirculation to the right ventricle. However, in the before-infarcted study, the third peaks are not obvious because the amount of tracer injected was half of that in the after-infarcted study. Therefore, the occurrence of the third peaks may be tracer-dependent. AIF is not computed by recirculation, or the effect is too small to consider.

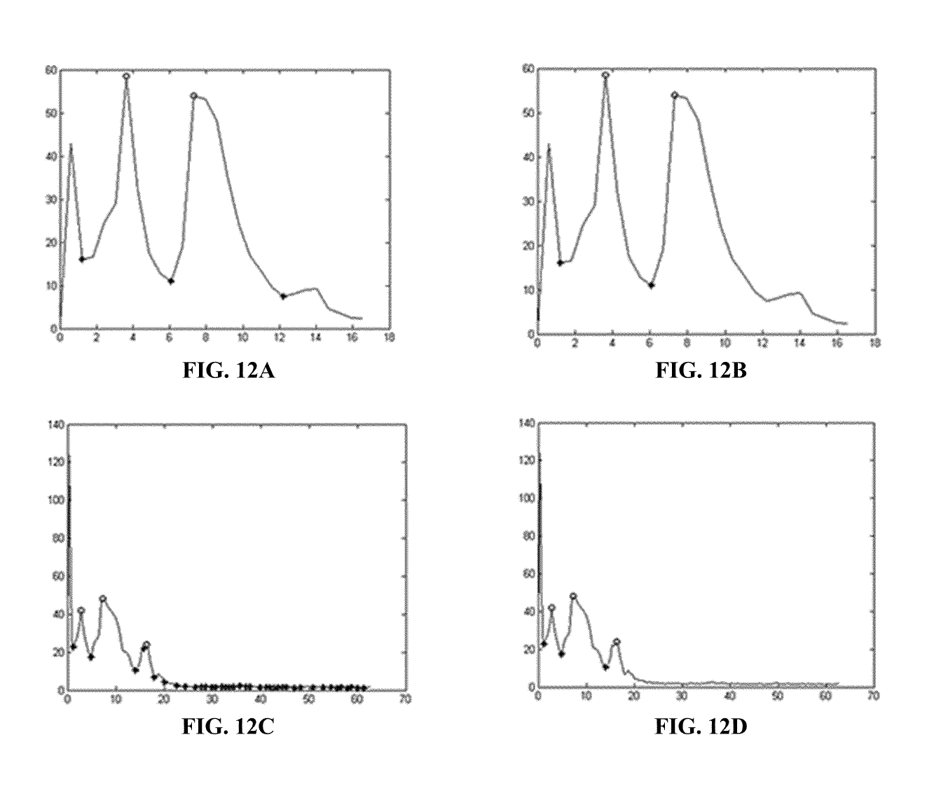

The automated processes for selecting peaks and valleys for both studies are shown in FIGS. 12A-12D. FIGS. 12A and 12C show a plot indicating potential peaks and valleys for the before and after infracted studies. The potential peaks and valleys were obtained using the methods described with respect to FIGS. 5A and 5B (providing the refined peaks 524 and refined valleys 534). The real peaks and valleys for these cases, obtained as described with respect to FIG. 5C, are shown in FIGS. 12B and 12D. Refined by the threshold requirements and the consistency features, two phases (right ventricle phase and left ventricle phase) or three phases (recirculation phase added) are automatically determined. No matter how many phases there are, the second phase mainly shows the blood pool in the left ventricle and associated major arteries, which are candidates for AIF.

Since in this project, the injection rate of both before-infarcted and after-infarcted studies did not change, while the amount of tracer in the after-infarcted study is twice more than that of the before-infarcted study, the maximum slope of AIF does not have a big difference, but the maximum enhancement increases (not exactly twice more).

In the before-infarcted study, the pixels are picked with the maximum enhancements bigger than the mean enhancement at the point of the peak in the second phase, whereas in the after-infarcted study, the pixels are picked with the maximum enhancements bigger than the mean plus standard variation of the enhancement at that point. For both studies, the AIF pixels selection are under the same condition that the maximum slopes are bigger than the mean slope at the point of the peak in the second phase. Through the process, the AIF is accurately and automatically selected. Using a similar method as in the first phase, the right ventricle and the associated major arteries are automatically segmented.

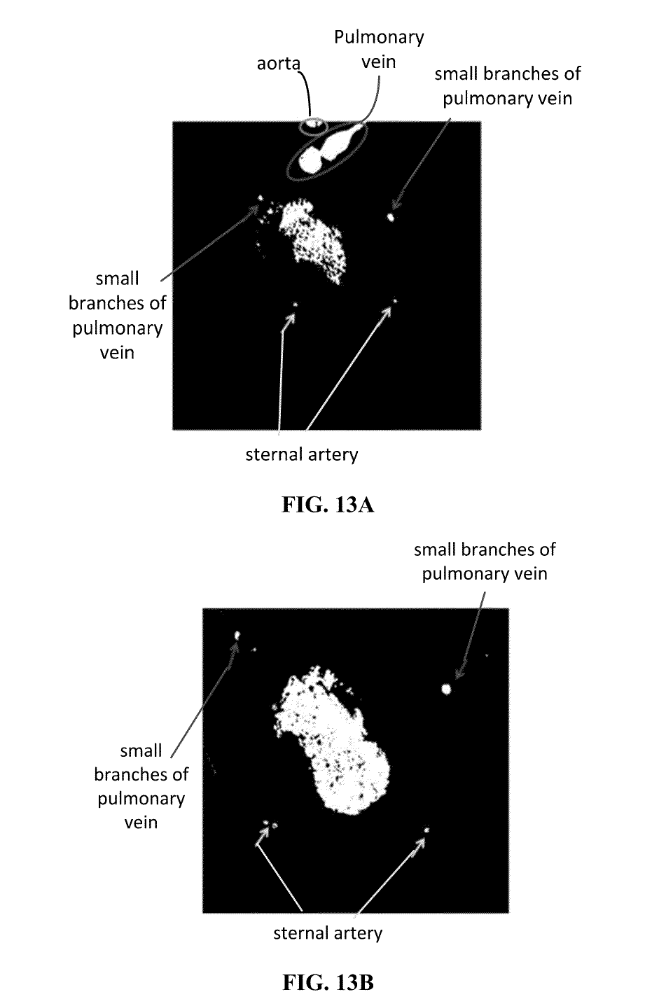

The results of the automated detection of AIF pixels are shown as binary images in FIGS. 13A and 13B. In both studies (before-infarcted and after-infarcted), the AIF pixels are located in the blood pool in pulmonary vein, left atrium, left ventricle, aorta, and the branches of aorta and pulmonary vein, which are blood supply areas to coronary arteries to feed the myocardium. Even the blood supply areas blocked by some parts of myocardium can be selected accurately.

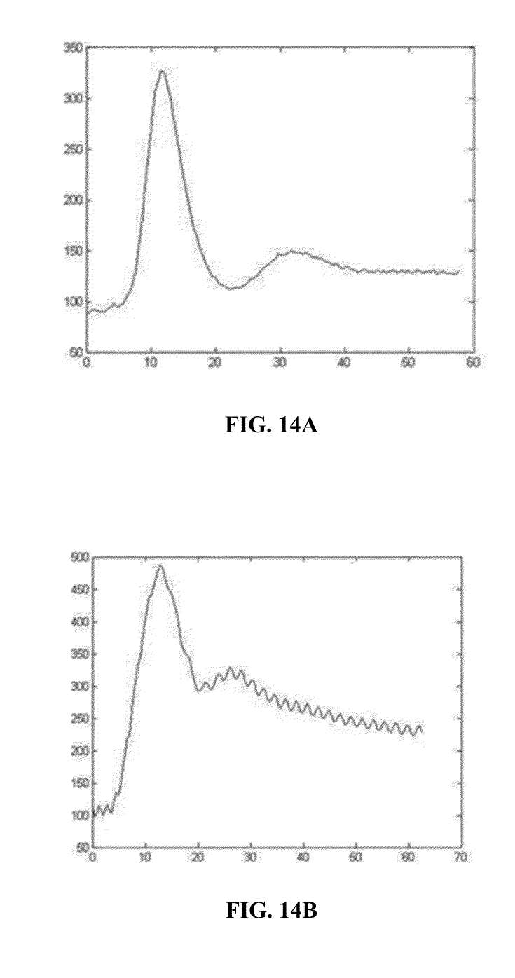

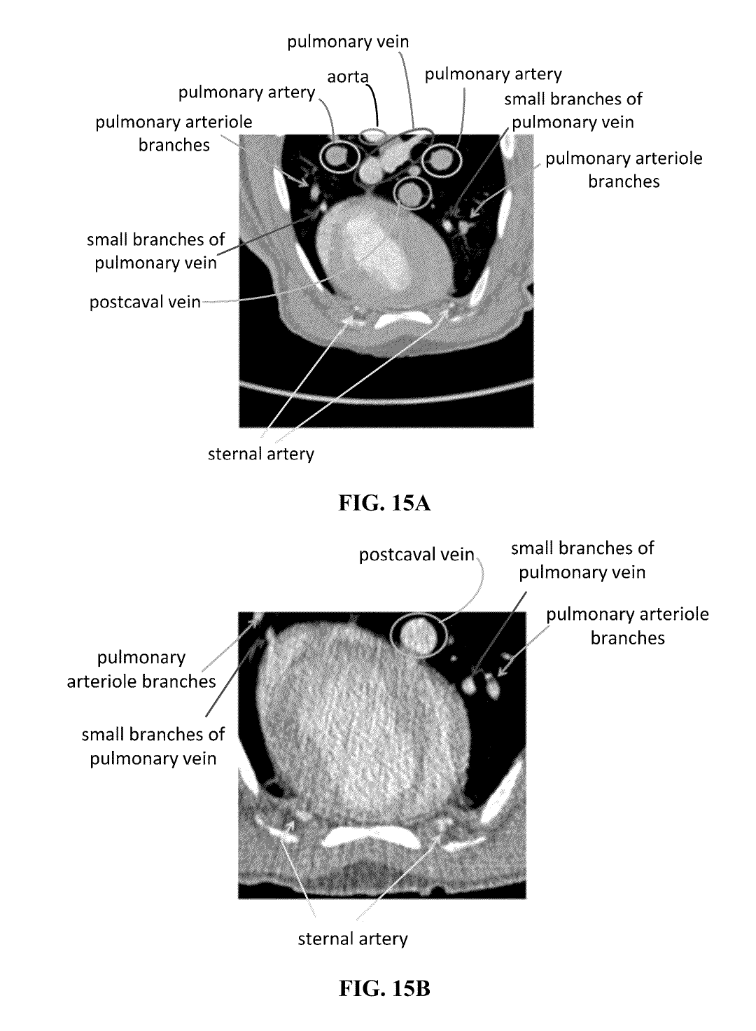

Despite the scattered distribution of the blood supply areas, the selection of AIF pixels is more efficient and more accurate than the manually selected ones. The average TACs, such as shown in FIGS. 14A and 14B, of the selected AIF pixels are smooth, which represents the uniform patterns of the bolus wash-in and wash-out processes in both studies. FIGS. 15A and 15B show the original 3D anatomical images. FIG. 15A is from the before-infarcted study and FIG. 15B is from the after-infarcted study. The anatomy is labeled in the images. Here, the aorta, pulmonary vein, pulmonary artery, postcaval vein, small branches of pulmonary vein, pulmonary arteriole branches, and the sternal artery (originating from the aorta) may be visible.

### Perfusion Maps and 3D Perfusion Volume Generation

Once AIF areas are selected, perfusion parametric maps are generated for each slice (e.g., position). Generally, perfusion maps are represented by myocardial blood flow (MBF). To obtain the perfusion maps, a maximum slope analysis, also referred to as upslope analysis, can be utilized. In the example study, the calculation process is simplified by three assumptions: first, perfusion tracer is neither metabolized nor absorbed by the tissue through which it traverses; second, it is an incompressive fluid dynamic process, which means that fluid flow-in equals to flow-out, corresponding to the interested tissues; third, a one compartment model is used by assuming that when mass accumulation of tracer is at the maximum in the tissue, the tracer in flow-out yields to zero. Hence, MBF can be represented as the ratio of the maximum slope of tissue time-attenuation curves s to the maximum arterial concentration:

$${[ \frac{d Q ( t )}{d t} ]}_{Max} = MBF \cdot {[ {C}_{artery} ( t ) ]}_{Max}$$

where Q(t) is the mass accumulation of tracer in the tissue (myocardium), and C_{artery} (t) is the tracer concentration in the AIF areas.

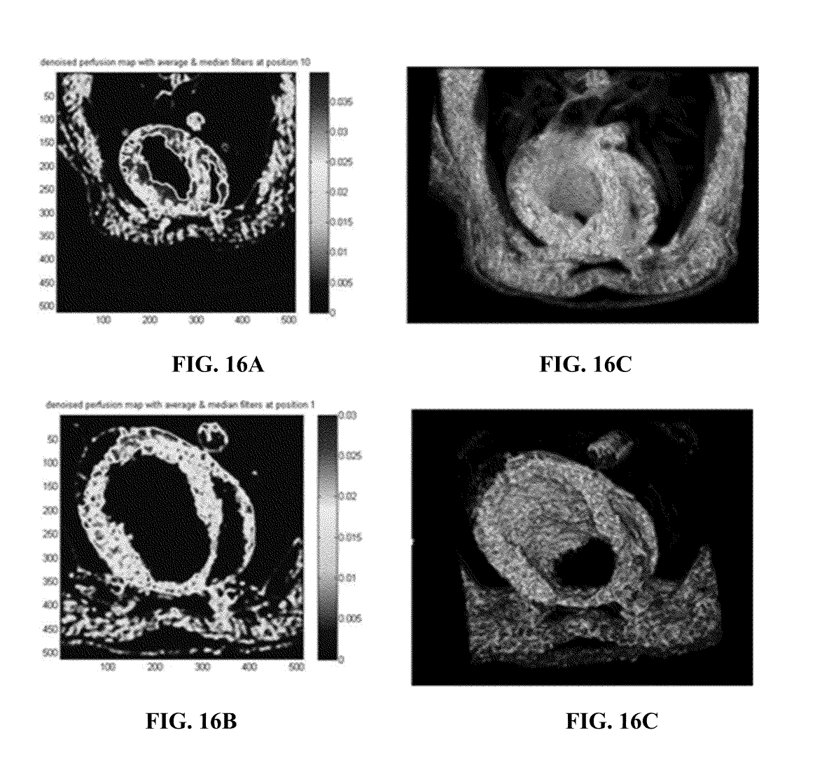

The MBF maps are generated, as shown in the perfusion maps of FIGS. 16A and 16B, to show the myocardial blood flow and distribution. By comparing the before (FIG. 16A) and after (FIG. 16B), it can be seen that there is normal enhancement in the inferoseptal wall, and dramatically reduced perfusion in the anterolateral wall, which is also much thinner. The 3D perfusion volume, as shown as FIGS. 16C and 16D, is reconstructed from series of MBF 2-D maps to anatomically and functionally assess myocardial physiological conditions.

### Example

### PET Abdominal Perfusion Studies

### PET Imaging in Gastrointestinal (GI) Perfusion

An example abdominal study is carried out illustrating the use of a PET perfusion study using an embodiment of automated AIF selection as described herein. In contrast to the CT myocardial perfusion studies, the abdominal studies were carried out using PET imaging with Cu⁶²-PTSM tracers. Four independent studies (Study 1, Study 2, Study 3, and Study 4) were performed on four ovine. In Study 1 and 3, the Cu⁶²-PTSM was with similar high radioactivity, and in Study 2 and 4, it was with similar low radioactivity.

### Animal Preparation

In these experiments, four adult 60-80 kg ovine were used for the PET abdominal perfusion studies after approval from IACUC. The studies were performed under a variety of cardiac output conditions.

### Microsphere Measurement

The microsphere studies were performed 20 minutes before each PET scan. Different colored microspheres were injected into the left ventricle during the five modes. Gold, samarium, ytterbium, europium and terbium color microspheres were used in the five modes—baseline, low continuous flow, high continuous flow, low induced pulse flow and high induced flow—respectively. The intestinal and renal tissue biopsies were harvested for the microsphere analysis after the study was terminated.

Radioactivity of Cu⁶²-PTSM was also tested to determine optimal radioactivity for the studies.

### PET Scan Imaging Protocol

PET/CT scans were performed using Siemens Biograph mCT (Siemens Molecular Imaging, Tennessee, US). The scanner is equipped with a 128 slice molecular CT and high resolution time-of-flight (TOF) PET with extended field-of-view (FOV). The subjects were positioned in head first-supine (HFS) orientation in the scanner. CT scans were implemented first through the whole body to optimize the region of interest (ROI), which locates from right kidney to small intestines, followed by the PET scans.

PET imaging involves a longer acquisition time than CT. Therefore, some important information during the dynamic process might be missing if the interval of each frame takes too long. However, if the interval is too short, the safety concern becomes a big issue due to the radioactive material exposure. In order to determine a better imaging protocol for PET perfusion studies, two groups of scans were performed with different frame durations, different scan time, but the same other settings.

In Study 1 and Study 2, the PET scans were performed over a period of 8 minutes with 30 seconds per frame (16 frames as total). 221 slices of images were obtained with 1 mm slice thickness. In the Study 3 and Study 4, the PET scans were performed over a period of 10 minutes with 10 seconds per frame (60 frames as total). 222 slices of images were obtained with 1 mm slice thickness. In the four studies, the Cu⁶²-PTSM was infused into the left ventricle through a peripheral intravenous tube around 30 seconds after the PET scans started. A 3 dimensional Gaussian filter with a full-width-half-maximum response of 5.0 mm was used as the kernel convolution for the later reconstruction. After each scan, the subjects were left inside the scanner for 40 minutes in order to let the radionuclides decay and be cleared out.

### Automated AIF Determination

Cu⁶²-PTSM becomes biologically trapped by tissues when it is injected into the body. Therefore, unlike the behavior inside arteries or blood pool areas, the Cu⁶²-PTSM experiences no wash-out process in the tissues. To address this scenario, the additional computations involving wash-out (e.g., 620 of FIG. 6) were included in the algorithm for automated AIF selection.

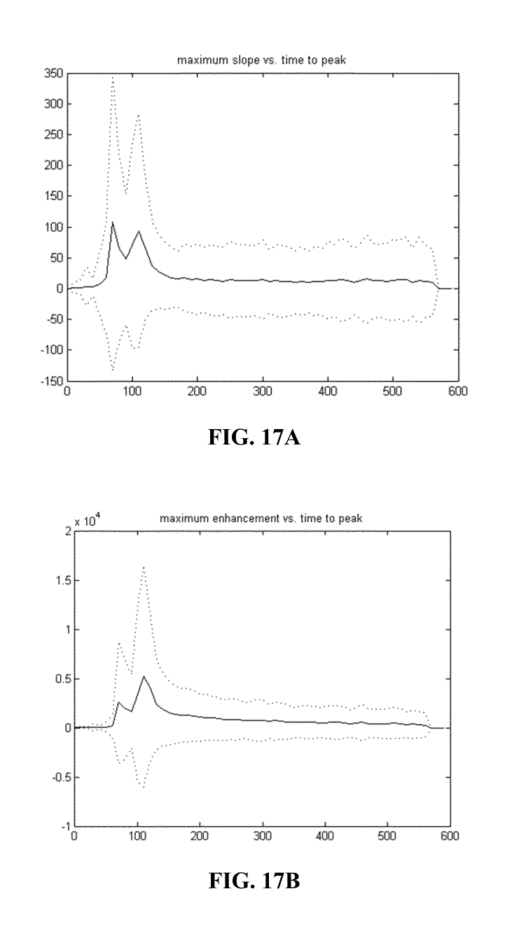

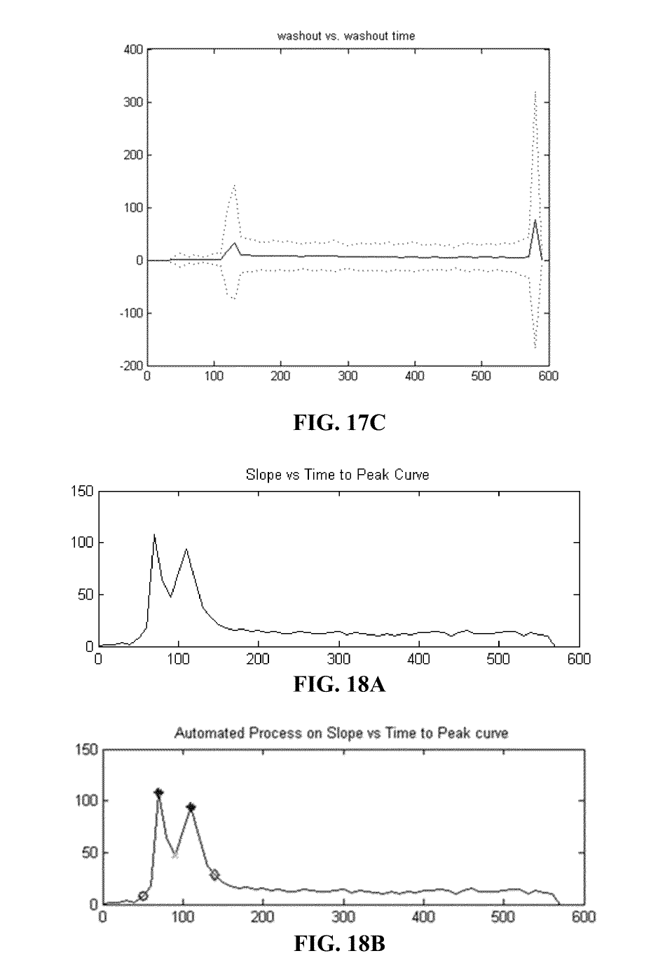

To extract the characteristic parameters and perform the pattern recognition (e.g., steps 310 and 320 of FIG. 3), the threshold constant α was set to 0.3 to provide an optimal threshold θ₁ for the wash-in parameters and wash-out parameters calculation. The five parameters were extracted: maximum enhancement, maximum slope, time-to-peak, wash-out slope, and time-to-wash-out. Three 2-D plots were generated: S vs. T curve (FIG. 17A), E vs. T curve (FIG. 17B), and W vs. T curve (FIG. 17C).

As shown in FIGS. 17A and 17B, there are two peaks on both the S vs. T curve and the E vs. T curve. The abdomen region was scanned from the right kidney (top) to the small intestines (bottom). Kidneys have very high metabolic activity. Therefore, the first peak represents the arteries and the associated branches, and the second is a result of tracer in the kidneys. As shown in FIG. 17C, on the W vs. T curve, since only the arterial phase has the wash-out process, the single peak is the expression of the artery in general.

For the peak and valley determination step (330 of FIG. 3; 516 of FIG. 5A), the slope derivative threshold, enhancement derivative threshold and wash-out derivative threshold were chosen based on the requirement that small noise should be removed completely.

According to the imaging protocol, the tracers infused process happened within 4 min, and after that, the tracers were either cleared up by the arterial system or trapped by tissues. Steady state was maintained during the rest of the scanning period. Therefore, all the automated calculation was executed in the period from 0 min to 4 min.

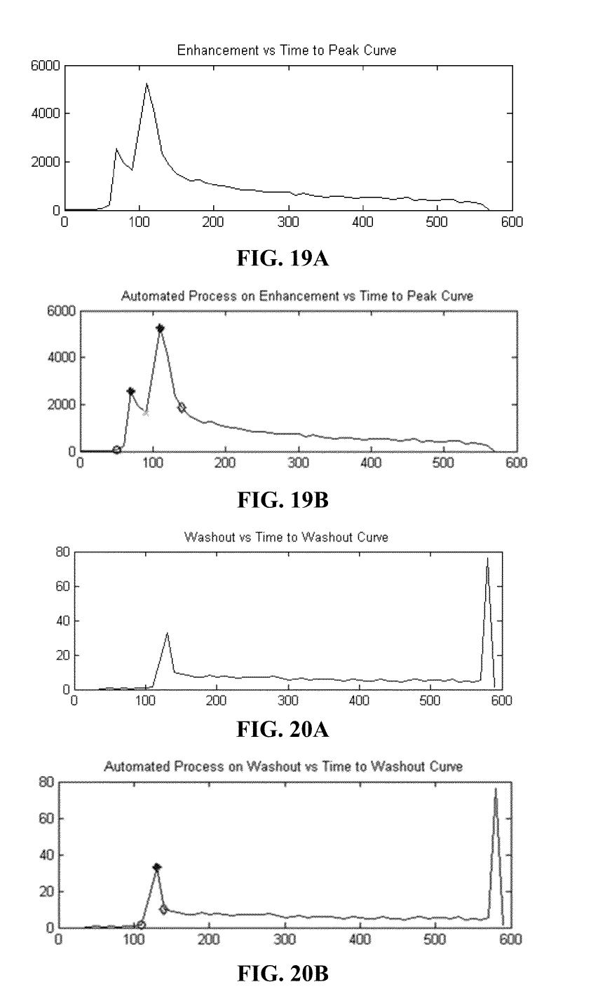

The automated processes for selecting characteristic points are shown in FIGS. 18-20. The arteries and kidneys phases were accurately selected. FIGS. 18A-18B show the automated process on the S vs. T curve where wash-in, wash-out, valleys and peaks are automatically determined. FIGS. 19A-19B and 20A-20B show the automated process for the E vs. T curve and W vs. T curve, respectively.

To pick the AIF, the results from the three plots were integrated. The selected pixels satisfied the following requirements: the maximum enhancement is bigger than the mean enhancement at the point of the peak (in the phase of interest) on the E vs. T curve, the maximum slope is bigger than the mean slope at the point of the peak (in the phase of interest) on the S vs. T curve, and the wash-out slope bigger than the mean wash-out slope at the point of the peak on the W vs. T curve. These selected pixels further meet the time requirements where the time to peak associated with these pixels is within the peaks (in the phase(s) of interest) on both E vs. T curve and S vs. T curve, and the time to wash-out is within the single peak on the W vs. T curve.

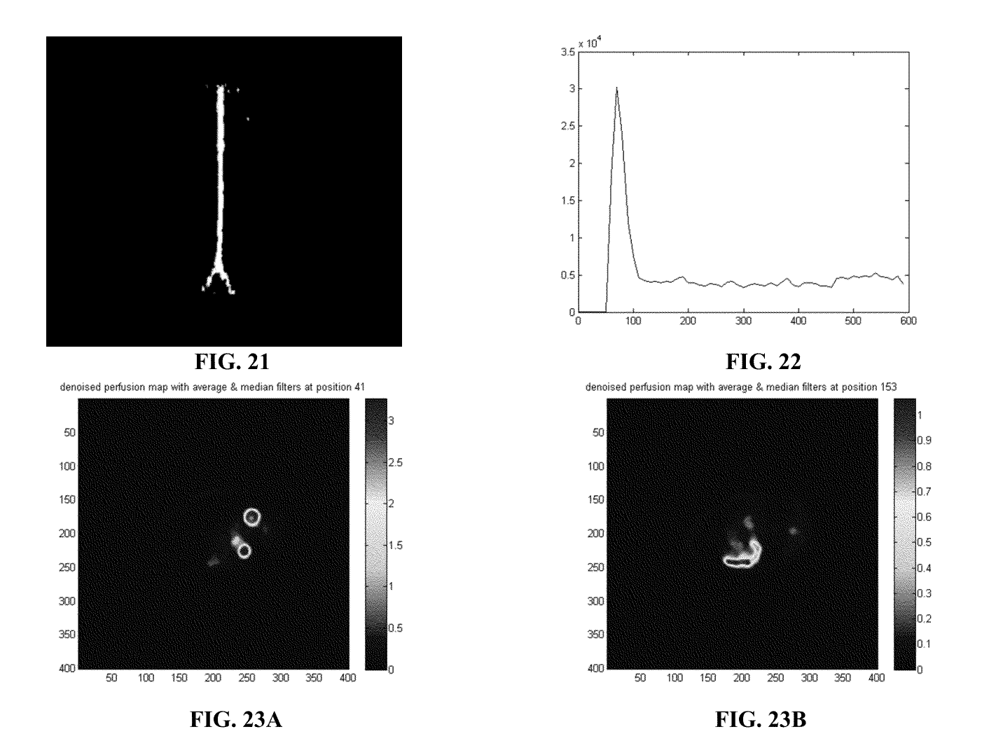

The result of the automated detection of AIF pixels is shown in a 3D binary image in FIG. 21. An intact and clear arterial system is shown in this figure: a main artery originated from aorta and then distributed into two branches. This artery system is the blood feeding areas for the entire abdomen. The average PET-TAC, as shown in FIG. 22, of the AIF pixels is smooth and represents the uniform patterns of the tracers wash-in and wash-out processes.

### Perfusion Maps Generation

Once AIF areas are selected, perfusion parametric maps are generated for each slice (e.g., position). To obtain the perfusion maps for each slice location, a trapped radiotracers model was applied. To calculate the time for the tracer washing into the arteries (i.e. “wash-in”), the time to maximum enhancement (as indicated on the PET-TAC of the arterial phase) is determined.

In Study 1 and Study 2, images were acquired every 30 seconds, and the interval between the tracer arrival to the maximum enhancement took 30 seconds. Therefore, the wash-in time was determined as 0.5 min. In Study 3 and Study 4, images were acquired every 10 seconds, and the interval between the tracer arrival to the maximum enhancement took 20 seconds (two 10 seconds). Therefore, the wash-in time was determined as ⅓ min.

FIGS. 23A and 23B show the generated perfusion maps of the kidneys and upper GI. The blood flows of kidneys, upper GI and lower GI match microsphere data well in general, which establishes the relationship between PET data and microsphere data (regarded as the “Gold Standard” study in tissue perfusion studies) and demonstrates that PET imaging is a good tool to be used in the abdominal perfusion studies

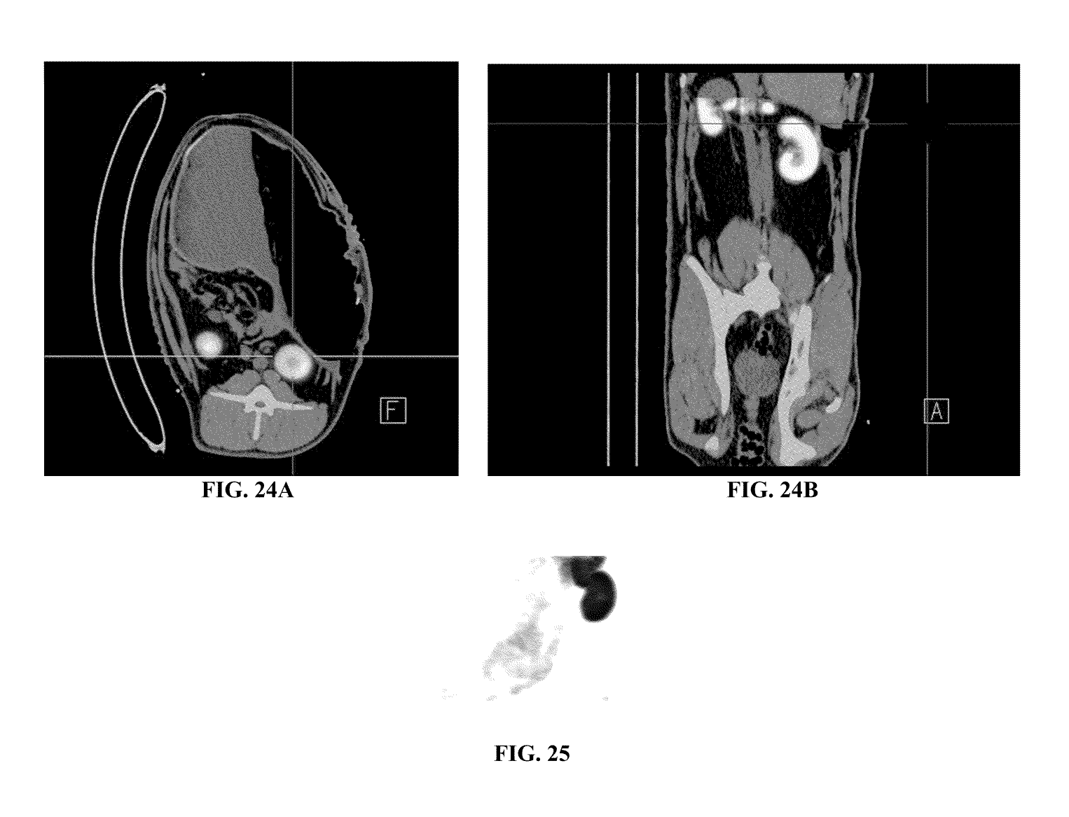

FIGS. 24A-24B show the fused perfusion maps with CT anatomy images and FIG. 25 shows the 3D perfusion volume. The registration of the two modalities provides the information for both anatomy and functionality of the tissues.

Certain techniques set forth herein may be described in the general context of computer-executable instructions, such as program modules, executed by one or more computers or other devices. Generally, program modules include routines, programs, objects, components, and data structures that perform particular tasks or implement particular abstract data types. Certain methods and processes described herein can be embodied as code and/or data, which may be stored on one or more computer-readable media. Certain embodiments of the invention contemplate the use of a machine in the form of a computer system within which a set of instructions, when executed, can cause the system to perform any one or more of the methodologies discussed above.

In some embodiments, the machine/computer system can operate as a standalone device. In some embodiments, the machine/computer system may be connected (e.g., using a network) to other machines. In certain of such embodiments, the machine/computer system may operate in the capacity of a server or a client user machine in server-client user network environment, or as a peer machine in a peer-to-peer (or distributed) network environment.

The machine/computer system can be implemented as a desktop computer, a laptop computer, a tablet, a phone, a server, or any other machine capable of executing a set of instructions (sequential or otherwise) that specify actions to be taken by that machine, as well as multiple machines that individually or jointly execute a set (or multiple sets) of instructions to perform any one or more of the methods described herein.

The computer system can have hardware including one or more central processing units (CPUs) and/or digital signal processors (DSPs), memory, mass storage (e.g., hard drive, solid state drive), I/O devices (e.g., network interface, user input devices), and a display (e.g., touch screen, flat panel, liquid crystal display, solid state display). Elements of the computer system hardware can communicate with each other via a bus.

When a computer system reads and executes instructions that may be stored as code and/or data on a computer-readable medium, the computer system performs the methods and processes embodied as data structures and code stored within the computer-readable medium.

Computer-readable media includes storage media in the form of removable and non-removable structures/devices that can be used for storage of information, such as computer-readable instructions, data structures, program modules, and other data used by a computing system/environment. By way of example, and not limitation, a computer-readable storage medium may include volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); or other media now known or later developed that is capable of storing computer-readable information/data for use by a computer system. “Computer-readable storage media” should not be construed or interpreted to include any carrier waves or propagating signals.

Furthermore, the methods and processes described herein can be implemented in hardware modules. For example, the hardware modules can include, but are not limited to, application-specific integrated circuit (ASIC) chips, field programmable gate arrays (FPGAs), and other programmable logic devices now known or later developed. When the hardware modules are activated, the hardware modules perform the methods and processes included within the hardware modules.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

It should be understood that the examples and embodiments described herein are for illustrative purposes only and that various modifications or changes in light thereof will be suggested to persons skilled in the art and are to be included within the spirit and purview of this application.

What is claimed is:

1. A system for performing automated determination of arterial input function (AIF) areas, comprising:
a characteristic parameter extractor extracting characteristic parameters from imaging data acquired to determine perfusion information about a subject;
a characteristic parameter map generator generating characteristic parameter maps to show relationships among the extracted characteristic parameters and converting the characteristic parameter maps to a plurality of two-dimensional plots; and
a tissue segmentation and AIF area determiner performing automated segmentation of non-AIF tissues and automated determination of AIF areas by automatically finding peaks and valleys of each phase of AIF areas on the plurality of two-dimensional plots.

2. The system according to claim 1, further comprising:
a perfusion parametric map generator generating perfusion parametric maps based on the automatically determined AIF areas and outputting the perfusion parametric maps for display.

3. The system according to claim 1, wherein the imaging data comprises imaging data acquired from positron emission tomography (PET), computed tomography (CT), single photon emission computed tomography (SPECT), ultrasound, luminescent, fluorescent, or magnetic resonance imaging (MRI).

4. The system according to claim 1, wherein the characteristic parameters extracted by the characteristic parameter extractor comprise maximum enhancement, maximum slope, and time-to-peak.

5. The system according to claim 4, wherein the plurality of two-dimensional plots comprises maximum slope vs. time-to-peak and maximum enhancement vs. time-to-peak.

6. The system according to claim 4, wherein the characteristic parameters extracted by the characteristic parameter extractor further comprise wash-out slope and time to wash-out.

7. The system according to claim 6, wherein the plurality of two-dimensional plots comprises wash-out slope vs. time to wash-out, maximum enhancement vs. time to peak, and maximum slope vs. time to peak.

8. The system according to claim 1, wherein the tissue segmentation and AIF area determiner comprises:
a peak-valley validator identifying peak candidates on the plurality of two-dimensional plots;
a valley estimator identifying valley candidates on the plurality of two-dimensional plots; and
a peak-valley determiner determining real peak points and real valley points from the peak candidates and valley candidates.

9. A method for performing automated determination of arterial input function (AIF) areas, comprising:
extracting characteristic parameters from imaging data acquired to determine perfusion information about a subject;
generating characteristic parameter maps to show relationships among the extracted characteristic parameters and converting the characteristic parameter maps to a plurality of two-dimensional plots; and
performing automated segmentation of non-AIF tissues and automated determination of AIF areas by automatically finding peaks and valleys of each phase of AIF areas on the plurality of two-dimensional plots.

10. The method according to claim 9, further comprising:
generating perfusion parametric maps based on the automatically determined AIF areas and outputting the perfusion parametric maps for display.

11. A computer-readable storage medium having instructions stored thereon that when executed by a computing device cause the computing device to perform a method comprising:
extracting characteristic parameters from imaging data of a subject for evaluating perfusion information of the subject;
performing pattern recognition to identify relationships between one or more of the characteristic parameters and generate two-dimensional (2D) plots from the relationships;
performing peak and valley determination with respect to the 2D plots; and
selecting pixels representing an arterial input function (AIF) area using the peak and valley determination for the 2D plots.

12. The medium according to claim 11, wherein extracting the characteristic parameters from the imaging data comprises extracting time to peak, maximum slope, and maximum enhancement.

13. The medium according to claim 12, wherein extracting the characteristic parameters from the imaging data further comprises extracting wash-out slope and time to wash-out.

14. The medium according to claim 11, wherein performing pattern recognition to generate the 2D plots comprises generating, for pixels of the imaging data, maximum slope vs. time to peak (S vs. T) curves and maximum enhancement vs. time to peak (E vs. T) curves.

15. The medium according to claim 14, wherein performing pattern recognition to generate the 2D plots further comprises generating, for pixels of the imaging data, wash-out vs. time to wash-out curves.

16. The medium according to claim 15, wherein performing peak and valley determination for the 2D plots comprises determining possible peak points in the 2D plots, estimating possible valley points in the 2D plots, and determining real peak points and real valley points from the possible peak points and the possible valley points.

17. The medium according to claim 14, wherein performing peak and valley determination for the 2D plots comprises determining possible peak points in the 2D plots, estimating possible valley points in the 2D plots, and determining real peak points and real valley points from the possible peak points and the possible valley points.

18. The medium according to claim 11, wherein selecting pixels representing the AIF area using the peak and valley determination for the 2D plots comprises:
for each pixel, if:
a maximum enhancement is greater than a mean enhancement at a point of a first peak on the E vs. T curve; and
a maximum slope is greater than a mean slope at a point of a first peak on S vs. T curve; and
a wash-out slope is greater than a mean wash-out slope at a point of a peak on the W vs. T curve; and
a time to peak is within the first peaks on the E vs. T curve and the S vs. T curve; and
a time to wash-out is within the peak on the W vs. T curve,
then assign the pixel as an AIF area;
else discard as being not the AIF area.

19. The medium according to claim 11, further comprising instructions that when executed by the computing device cause the computing device to perform the method further comprising:
generating a perfusion parametric map using the pixels representing the AIF area; and
displaying the perfusion parametric map.

20. The medium according to claim 11, wherein the imaging data comprises imaging data acquired from positron emission tomography (PET), computed tomography (CT), single photon emission computed tomography (SPECT), ultrasound, luminescent, fluorescent, or magnetic resonance imaging (MRI).
